(19) United States
(12) Patent Application Publication
(10) Pub. No.: US 2025/0000662 A1
(43) Pub. Date: Jan. 2, 2025

(54) STEMLESS, CONVERTIBLE, HUMERAL SHOULDER PROSTHESIS

(71) Applicant: Shoulder Innovations, Inc., Grand Rapids, MI (US)

(72) Inventors: Robert J. Ball, West Olive, MI (US); Donald E. Running, Missoula, MT (US)

(21) Appl. No.: 18/883,873

(22) Filed: Sep. 12, 2024

Related U.S. Application Data
(63) Continuation of application No. PCT/US2023/015740, filed on Mar. 21, 2023.
(60) Provisional application No. 63/322,201, filed on Mar. 21, 2022.

(51) Int. Cl. A61F 2/40; A61F 2/30

(52) U.S. Cl. CPC A61F 2/4014; A61F 2002/30878; A61F 2002/4022; A61F 2002/4085

(57) ABSTRACT

In some aspects, a shoulder implant with improved joint mobility, includes a backing component, a neutral non-inclined bearing component, and a peg. The neutral non-inclined bearing component is located on a concave side of the backing component. The bearing component is made from a different material than the backing component and is configured to touch a glenosphere having a center of rotation, wherein the bearing component is substantially radially symmetrical. The peg extends from the backing component and is configured to be connected to a humeral stem. The longitudinal axis of the peg of the backing component is offset from the center of rotation of the glenosphere and the longitudinal axis of the backing component is aligned with the center of rotation of the glenosphere.

### CROSS-REFERENCE TO RELATED APPLICATIONS

This application is a continuation of International Application No. PCT/US2023/015740, filed on Mar. 21, 2023, which claims the benefit of the filing date of U.S. Provisional Patent Application No. 63/322,201, filed Mar. 21, 2022. The disclosure of each of the above applications is incorporated herein by reference in its entirety.

### BACKGROUND

Shoulder Replacement is a commonly performed medical procedure for treatment of osteoarthritis, rheumatoid arthritis, as well as for treatment of certain deformities related to oncological indications as well as trauma. There are two primary types of articulations available to surgeons for treatment: anatomic and reverse. With anatomic, the surgeon replaces the articular surfaces with industrial materials such that the articulating surfaces are substantially the same shape as the natural anatomy. A stem can be commonly fixed inside the canal of the humerus, a metallic articular head can be rigidly fixed to the proximal aspect of the same, the articular head having a convex articular surface adapted to articulate with the glenoid implant. The glenoid implant can include on its back side (medial side) certain pegs or posts or fins adapted to be rigidly fixed within the glenoid fossa of the scapula and on its front side a concave or flat articular surface adapted to articulate with the humeral head of the humeral implant.

When a reverse prosthesis is used, the articular surface is reversed in that the metallic ball is rigidly fixed to the glenoid fossa of the scapula, and the concave articular surface is rigidly fixed to the humeral bone, thereby reversing the fashion of articulation of the prosthesis.

The surgeon chooses between the two types of prostheses by assessing a number of conditions of the patient including level of pain, patient activity level, deformity or severity of the boney degradation, the strength of surrounding soft tissues, and present or absence of prior surgery, and particularly the health and strength of the rotator cuff muscle and tendon. Disease of the rotator cuff is common among patients with arthritis of the shoulder. In this circumstance, it is commonly observed that the absence of insufficiency of the rotator cuff leads to a condition where the anatomic shoulder replacement prosthesis is not sufficiently stabilized by surrounding soft tissue. In this case, a reverse shoulder replacement prosthesis can be preferred in some cases due to the higher inherent stability of the articulation. In addition, the reverse prosthesis can advantageously utilize the remaining muscles in a way they can be more effective in the absence of the other soft tissue structures by adjusting the position of the articular surfaces within the joint.

It is not uncommon that a surgeon selects to use an anatomic prosthesis and is provides effective treatment to the patient though the shoulder replacement operation. However, over time and during use of the prosthesis, the patient's rotator cuff complex can become insufficient, tear, or generally be diseased such that it can no longer perform its function associated with normal joint kinematics. In this case, the surgeon can elect to perform a second operation to remove the anatomic prosthesis and replace the anatomic prosthesis with a reverse prosthesis.

Several attempts have been made to attempt to address the need of conversion of the articular surface without interruption of the fixation. Primarily, these are created using a two (or more) system, where there is a metallic fixation component which is rigidly fixed to the glenoid fossa, and a polyethylene (PE) articular component which is secondarily fixed to the metallic component, and provides the concave articular surface adapted to articular with the humeral prosthesis. While referred to herein as a PE component, some embodiments do not require the use of polyethylene and can be made of other biocompatible materials depending on the desired clinical result. The PE component is commonly fixed to the metallic fixation component by conventional industrial techniques such as snap fit mechanisms, snap rings, compression pins, overmolding of the PE and other such means.

A challenge of this particular articulation in some cases is that the glenoid fossa is relatively small, and commonly there is much reduced presence of bone in patients with arthritis. In this context, the surgeon has limited positioning and bone to work with in order to fit within the patient. In addition, the surgeon must be careful not to overstuff the joint, meaning implant components that move the new articulating surface far from its original position such that the soft tissues is unnaturally tensioned, which can lead to instability, accelerated where, soft tissue failure, pain, reduced range of motion, or fracture of the prosthesis and surrounding bone. Facing these conditions, the prosthesis typically needs to be designed to remain relatively thin (commonly, 1 piece, where PE glenoid implants typically have a 4 mm thick articular surface). In order to design these modular components, there can be little additional packaging space provided into which to fit the attachment mechanisms necessary for use without adversely affecting the performance of the overall joint replacement procedure. Thus, typically, these designs lead to “over-optimization” of the fixation and articular portions in order to provide sufficient attachment mechanisms such that either: the PE is too thin to be sufficiently strong, the metallic components are too small to provide sufficient fixation, or the overall mechanism is insufficiently rigid causing there to be secondary wear surfaces, and generation of wear particles leading to PE disease.

A problem that can exist is that in the case where the surgeon wants to change the prosthesis type, the anatomic prosthesis is commonly well fixed and adapted to the patient's body such that removal of the prosthesis can be very destructive and leave natural bone remaining that is perhaps insufficient to support the fixation of the reverse prosthesis. What is needed is a prosthesis system that provides a means by which the articulating surfaces of the implant can be exchanged such that the anatomic surfaces can be converted to reverse surfaces, while not exchanging the fixation components.

What is also needed is a simple means by which the surgeon can implant an inset anatomic articulating glenoid implant whereby at a later date, can remove the anatomic articulating surface and replace it with a reverse articulating surface such that the primary means of fixation remains well fixed in the glenoid fossa at the moment of articular exchange.

Furthermore, conventional systems can change the inclination angle of the humerus by offering bearing components (e.g., polyethylene inserts) that are more and more inclined, thus medializing the center of rotation of the glenosphere and overstuffing the joint. Systems that can change the inclination angle of the humerus without changing the center of rotation of the glenosphere and/or overstuffing the joint to provide increased range of motion are needed.

### SUMMARY

In some aspects, the techniques described herein relate to a shoulder implant with improved joint mobility, including: a backing component; a neutral non-inclined bearing component on a concave side of the backing component, the bearing component made from a different material than the backing component and configured to touch a glenosphere having a center of rotation, wherein the bearing component is substantially radially symmetrical; and a peg extending from the backing component and configured to be connected to a humeral stem; wherein the longitudinal axis of the peg of the backing component is offset from the center of rotation of the glenosphere, wherein the longitudinal axis of the backing component is aligned with the center of rotation of the glenosphere.

In some aspects, the techniques described herein relate to a shoulder implant, wherein the backing component is inclined by an angle of between about 1.25 degrees and about 30 degrees with respect to a corresponding attachment surface of a humeral head.

In some aspects, the techniques described herein relate to a shoulder implant, wherein the backing component is inclined by an angle of between about 2.5 degrees and about 12.5 degrees with respect to a corresponding attachment surface of a humeral head.

In some aspects, the techniques described herein relate to a shoulder implant, wherein the glenoid implant when inserted into a patient does not change the center of rotation of the glenosphere.

In some aspects, the techniques described herein relate to a shoulder implant, configured for anatomic positioning.

In some aspects, the techniques described herein relate to a shoulder implant, configured for reverse positioning.

In some aspects, the techniques described herein relate to a shoulder implant, wherein the bearing component has a depth of less than about 5.5 mm.

In some aspects, the techniques described herein relate to a shoulder implant, wherein the bearing component has a depth of between about 3 mm and about 5 mm.

In some aspects, the techniques described herein relate to a shoulder implant, wherein the bearing component includes a plastic material.

In some aspects, the techniques described herein relate to a shoulder implant, wherein the plastic material includes polyethylene.

In some aspects, the techniques described herein relate to a shoulder implant, wherein the backing component includes a metal material.

In some aspects, the techniques described herein relate to a shoulder implant, wherein the peg includes a Morse taper.

In some aspects, the techniques described herein relate to a shoulder implant, wherein at least a portion of an inferior-facing surface of the backing component configured to come in contact with a prepared humeral surface includes an oval or elliptical cross-section, at least a portion of the inferior-facing surface of the bearing component includes a circular cross-section, and at least a portion of the superior-facing surface of the backing component includes a circular cross-section, the portion of the inferior-facing surface of the bearing component and the superior-facing surface of the backing component configured to snap or otherwise mate with each other.

In some aspects, the techniques described herein relate to a kit 1, wherein the plurality of implants includes a first implant and a second implant, wherein the backing component of the first implant is inclined by a first angle, wherein the backing component of the second implant is inclined by a second angle different from the first angle, wherein the center of rotation of the glenosphere of the first implant and the second implant are in the same location.

In some aspects, the techniques described herein relate to a method of implanting a shoulder implant, including: reaming a cavity in the glenoid surface; sizing a first implant including a first angulation with respect to a stem, wherein the implant includes a neutral bearing component and an inclined backing component including a first incline angle with respect to a corresponding attachment surface of a humeral head; sizing a second glenoid implant including a second angulation with respect to the stem, wherein the glenoid implant includes a neutral bearing component and an inclined backing component including a second incline angle with respect to a corresponding attachment surface of a humeral head; implanting the second glenoid implant in the glenoid cavity; wherein the center of rotation of the glenosphere does not change between the first glenoid implant and the second glenoid implant, allowing for adjustment of version and inclination without changing the center of rotation.

In some aspects, the techniques described herein relate to a humeral prosthesis, including: a proximal end including at least three radially outwardly extending fins; a stem extending distally from the proximal end to a distal end, wherein the proximal end includes a proximal end diameter defined by a circle contacting radial outward-most proximal tips of the at least three radially outwardly extending fins, wherein the distal end includes a distal end diameter, wherein the proximal end diameter is between about 30 mm and about 50 mm, wherein the distal end diameter is between about 5 mm and about 6 mm, and wherein a ratio of the proximal end diameter and the distal end diameter is between about 5 and about 9.

In some aspects, the techniques described herein relate to a humeral prosthesis, wherein the ratio is between about 5.5 and about 8.5.

In some aspects, the techniques described herein relate to a humeral prosthesis, wherein the proximal end includes a bowl-shaped concavity.

In some aspects, the techniques described herein relate to a humeral prosthesis, including a central aperture in the proximal end configured to mate with a reverse component.

### BRIEF DESCRIPTION OF THE DRAWINGS

FIG. 1 is a side perspective view of an embodiment of a convertible humeral prosthesis, according to some embodiments of the invention.

FIG. 2 is a bottom perspective view of the embodiment shown in FIG. 1;

FIG. 3 is a top view of the embodiment shown in FIG. 1 also showing an aperture in the center of the superior facing surface of the neck of the prosthesis and configured to be utilized as a reverse adapter for, in some cases, facilitating placement or removal of a spherical ball on the humeral head.

FIG. 4 is a side view of the embodiment shown in FIG. 1;

FIG. 5 is another side view of the embodiment shown in FIG. 1, also illustrating channel of the reverse adapter aperture extending within the neck and stem.

FIG. 6 is an inverted perspective view, also illustrating medial groove along the neck and stem of the prosthesis.

FIG. 7 is another perspective view of the prosthesis shown in FIG. 1.

FIG. 7A is a side perspective view of another embodiment of a convertible humeral prosthesis, according to some embodiments of the invention.

FIG. 7B is a bottom perspective view of the embodiment shown in FIG. 7A;

FIG. 7C is a top view of the embodiment shown in FIG. 7A also showing an aperture in the center of the superior facing surface of the neck of the prosthesis and configured to be utilized as a reverse adapter for, in some cases, facilitating placement or removal of a spherical ball on the humeral head.

FIG. 7D is a side view of the embodiment shown in FIG. 7A;

FIG. 7E is another side view of the embodiment shown in FIG. 7A, also illustrating channel of the reverse adapter aperture extending within the neck and stem.

FIG. 7F is an inverted perspective view, also illustrating medial groove along the neck and stem of the prosthesis.

FIG. 7G is another perspective view of the prosthesis shown in FIG. 7F.

FIGS. 8-11 illustrate various views of another embodiment of a humeral replacement prosthesis.

FIG. 12 is an illustration of one potential embodiment of the invention that depicts a conventional glenoid implant with an offset bearing component.

FIG. 13 is an illustration of one embodiment of the offset backing component allowing a 2.5 degree inclination.

FIG. 14 is an illustration of one embodiment of the offset backing component allowing a 7.5 degree inclination.

FIG. 15 illustrate the use of one embodiment of the offset backing component allowing a 12.5 degree inclination.

FIG. 16A illustrates one embodiment of a stemmed humeral implant.

FIG. 16B illustrates one embodiment of a stemless humeral implant.

FIG. 17A illustrates a side view of the stemmed humeral implant of FIG. 16A.

FIG. 17B illustrates a side view of the stemless humeral implants of FIG. 16B.

FIG. 18A illustrates a cross-sectional side view of the stemmed humeral implant of FIG. 16A.

FIG. 18B illustrates a cross-sectional side view of the stemless humeral implant of FIG. 16B.

FIG. 19A illustrates the stemmed humeral implant of FIG. 16A with a mounting base attached to the stemmed humeral implant.

FIG. 19B illustrates the stemless humeral implant of FIG. 16B with a glenosphere with mounting base attached to the stemless humeral implant.

FIG. 19C illustrates the stemmed humeral implant of FIG. 19A, where the glenosphere is mounted to the mounting base of the stemmed implant.

FIG. 19D illustrates the stemless humeral implant of FIG. 19B, without a glenosphere, and with the mounting base attached to the stemless humeral implant.

FIG. 20A is a perspective view of the stemless implant of FIG. 16B.

FIG. 20B is a side view of the stemless implant of FIG. 20A.

FIG. 20C is a top view of the stemless implant of FIG. 20B.

FIG. 20D is a side cross-sectional view of the stemless implant of FIG. 20C taken along line A-A.

FIG. 20E is a detailed view of a portion of the view of FIG. 20D.

FIG. 20F is another side view of the stemless implant of FIG. 20B.

FIG. 20G is a side view of the stemless implant of FIG. 20C taken along line D-D.

FIG. 20H is a side cross-sectional view of the stemless implant of FIG. 20C taken along line C-C.

FIG. 21 is a side view of a broach being used to prepare a humerus to receive one of the stemmed or stemless implants described herein.

FIG. 22 is another side view of the broach of FIG. 21 being used to prepare the humerus to receive one of the stemmed or stemless implants described herein.

FIG. 23 is a perspective view of the humerus of FIG. 22 after it has been prepared to receive one of the stemmed or stemless implants described herein.

FIG. 24 is a side view of the humerus of FIG. 23 with a stemmed humeral implant implanted therein.

FIG. 25 is a cross-sectional view of the humerus and stemmed humeral implant of FIG. 24.

FIG. 26 is a side view of the humerus and stemmed humeral implant of FIG. 23 with a glenosphere attached to the stemmed humeral implant.

FIG. 27 is a side view of the humerus of FIG. 23 with a stemless humeral implant implanted therein.

FIG. 28 is a side view of the humerus and stemless humeral implant of FIG. 27 with a glenosphere attached to the stemless humeral implant.

FIG. 29 is a side view of the humerus of FIG. 23 showing both a stemmed and stemless implant implanted therein for comparative purposes.

FIG. 30 is a method of stemless humeral implant implantation.

FIG. 31 illustrates one embodiment of a stemmed humeral implant system.

FIG. 32A illustrates an exploded view of another embodiment of a stemmed humeral implant system.

FIG. 32B illustrates an exploded view of one embodiment of a stemless humeral implant system.

### DETAILED DESCRIPTION

In particular, some embodiments of the invention are focused on advantageously exchanging the articular surface of the glenoid from a concave shape to a convex shape, without removing the components or interface having to do with fixation of the implant into the glenoid fossa, by utilizing a convertible humeral prosthesis.

In some embodiments, embodiments of the invention can be used or modified with use with particular advantages of using inset glenoid fixation technology in anatomic shoulder arthroplasty, such as described, for example, in U.S. Pat. No. 8,007,538 to Gunther, which is hereby incorporated by reference in its entirety. In some embodiments, an inset method includes identifying a patient having a glenoid surface; reaming a cavity into the glenoid surface; and inserting a glenoid implant having a body and a single, radially symmetric central peg oriented along a central axis of the implant, the body having a bearing surface on a peripheral edge thereof into the cavity, such that at least a portion of a peripheral edge of the body is inset with respect to the cavity and resides below the adjacent glenoid surface and the portion residing below the adjacent glenoid surface is circumferentially surrounded by cortical bone of the glenoid.

What is further described are methods by which the surgeon can achieve the use of the inset glenoid technology with an anatomic articulation, while after having the ability to convert the technology to a reverse articulation, without requiring removal the rigid fixation between the inset fixation and the scapula bone (in other words, allowing the rigid fixation support between the inset fixation and the scapula bone to remain in place during conversion from an anatomic to a reverse prosthesis).

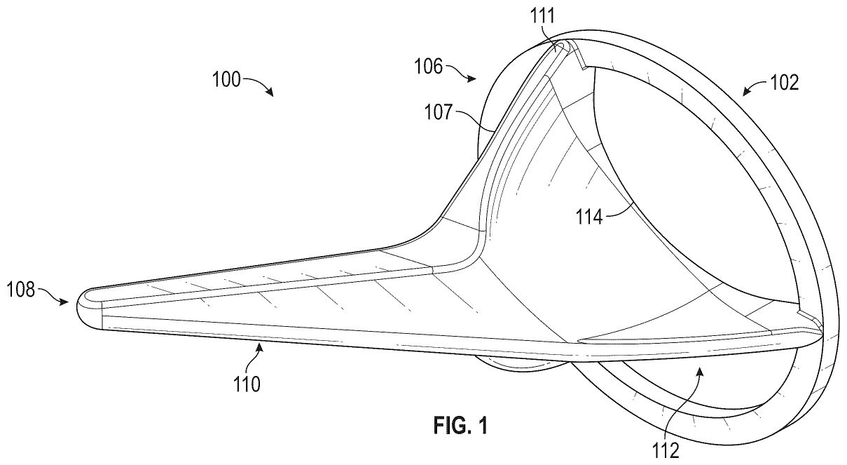

FIGS. 1-7 illustrate various views of a humeral replacement prosthesis 100, according to some embodiments of the invention. The prosthesis 100 can optionally include a proximal ring element 102, such as a collar (which can be configured to be attached to a spherical ball head portion, not shown) with a peripheral edge 103 that can be annular such as circular as shown, with an inferior-facing surface that can be optionally recessed (including a convex or flat bowl-like shape) and include a cavity, or inline in other embodiments. The humeral prosthesis 100 can include a superior-facing surface 114 of the proximal end 106, and be concave and bowl-shaped as shown with the base and deepest portion of the bowl proximate the center of the superior facing surface 114 of the proximal end 106 such that there is a space 113 between the inferior-facing surface of the humeral head portion (not shown) and the superior-facing surface 114 of the proximal end 106 of the prosthesis 100. In some embodiments the depth of the bowl at the center of concave curvature is between about 3 mm and about 7 mm, such as between about 4 mm and about 6 mm deep, such as about 5 mm deep. The proximal end 106 of the prosthesis 100 can include spaced-apart flanges 107, such as three flanges 107 spaced equally apart, with vertices 111 of the flanges 107 extending radially outwardly while becoming narrower from the center of the proximal end 106 of the prosthesis 100. In some cases, a tri-flange design can provide for improved stability and rotation prevention, among other advantages. Extending distally from the proximal end 106 at an angle to the longitudinal axis of the proximal end/neck 106 is the stem 110 and distal end 108 of the prosthesis 100.

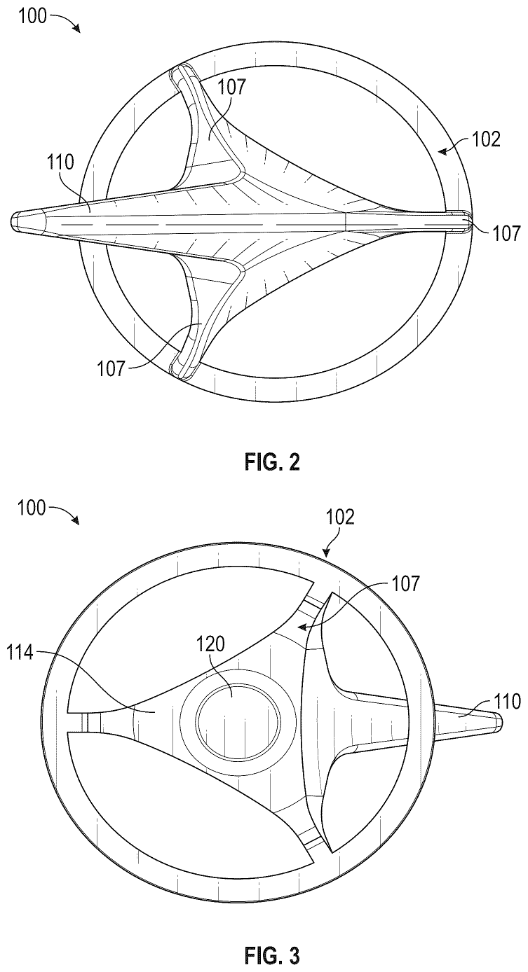

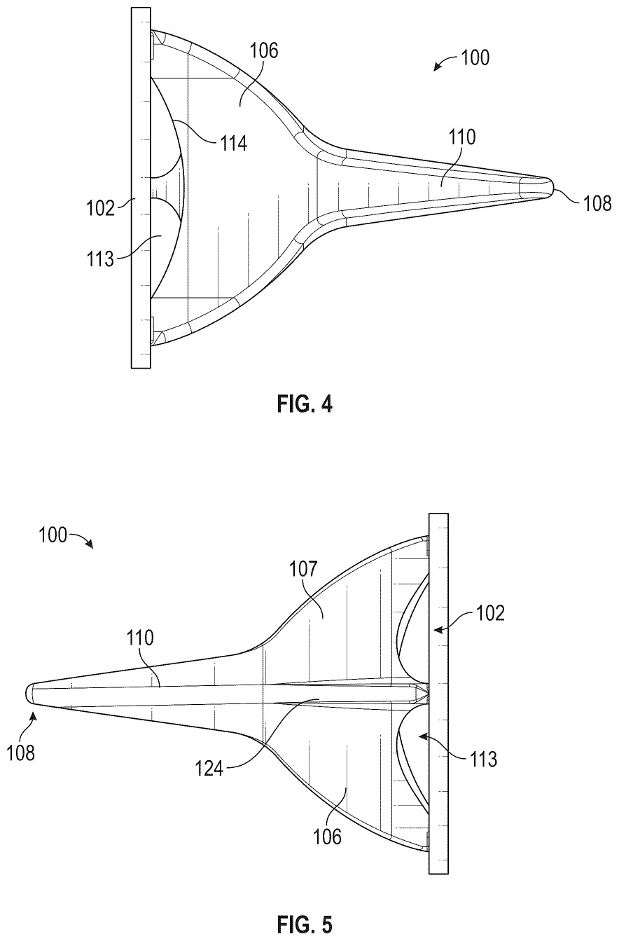

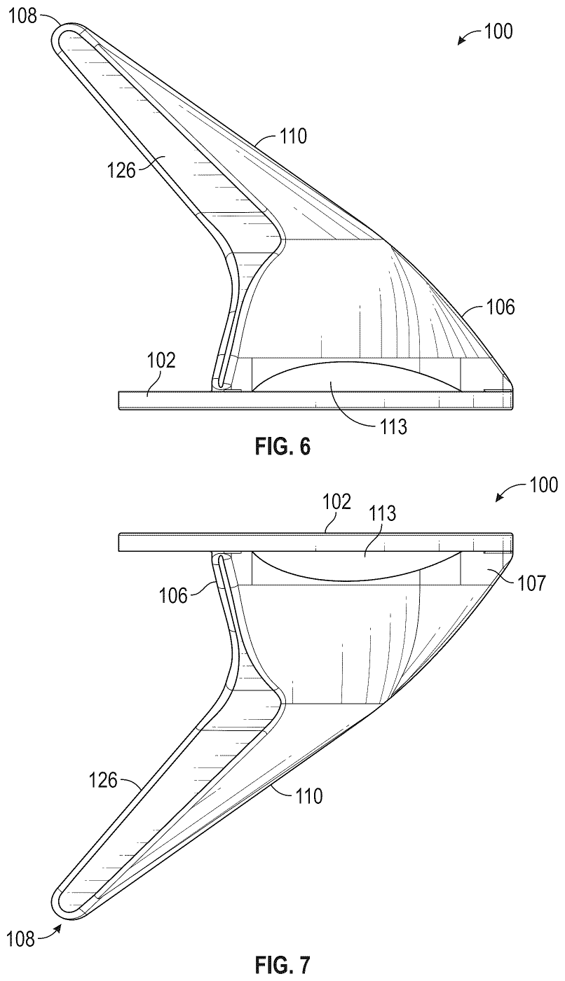

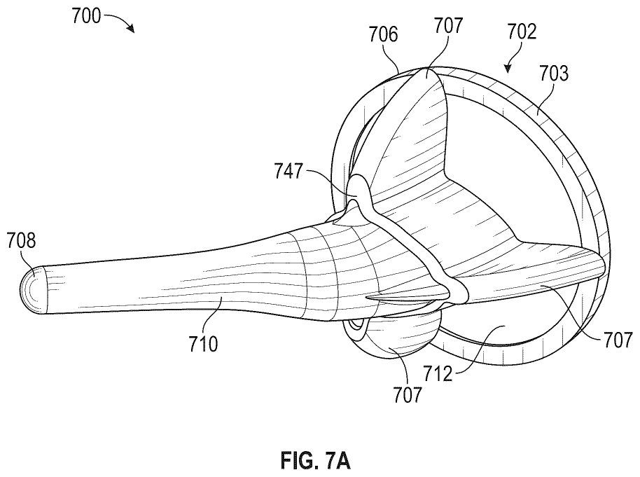

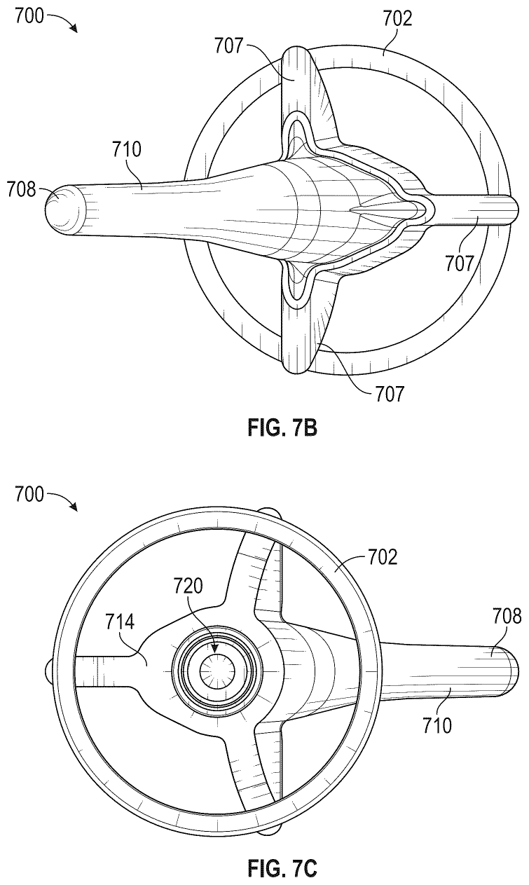

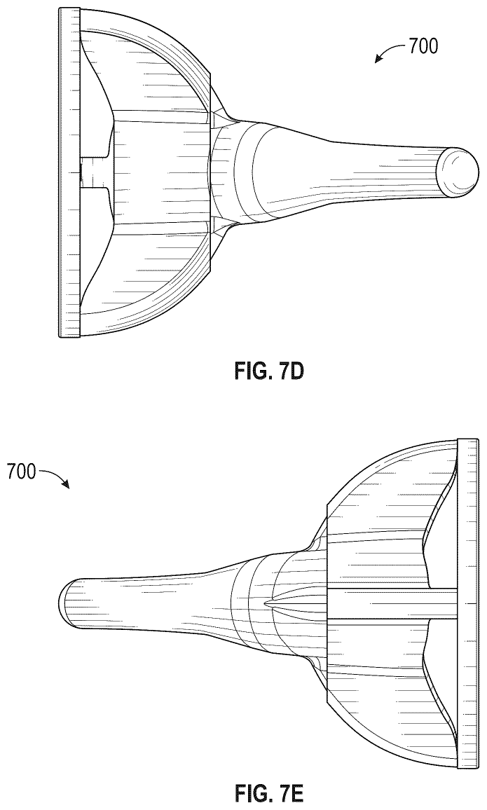

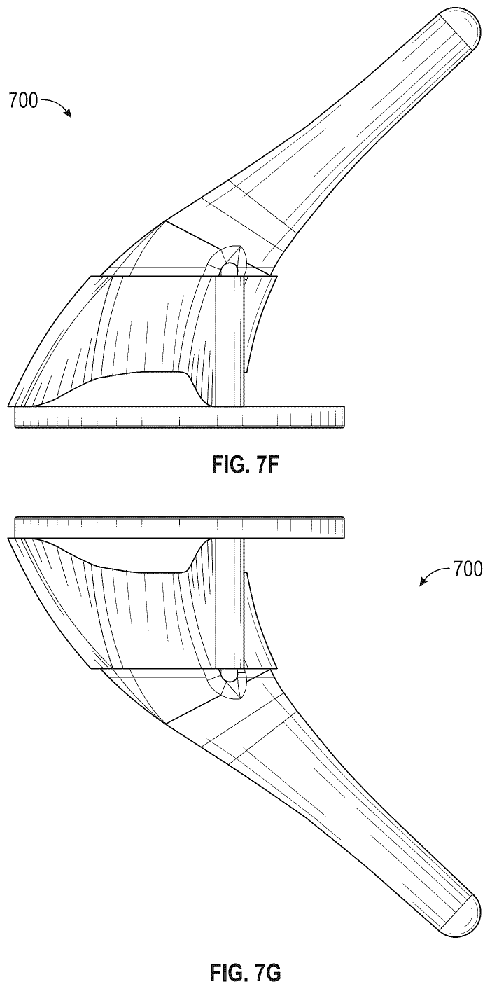

In some embodiments, the stem 110 and distal end 108 of the prosthesis has reduced thickness, e.g., tapering to no more than about 6 mm, 5.5 mm, 5 mm, 4.5 mm, 4 mm, or less in width dimension, such as between about 5.5 mm and about 6 mm, and ranges incorporating any two of the foregoing values, to advantageously reduce the quantity of native humerus bone needed to be removed, and allows for a stem 110 that is 3-4 mm or longer than a conventional stem 100 and allows for the majority of the fixation to occur in the proximal portion (e.g., neck 106) of the prosthesis. FIG. 1 is a side perspective view of an embodiment of the prosthesis; FIG. 2 is a bottom perspective view of the embodiment shown in FIG. 1; FIG. 3 is a top view of the embodiment shown in FIG. 1 also showing an aperture 120 in the center of the superior facing surface 114 of the neck 106 of the prosthesis and configured to be utilized as a reverse adapter for, in some cases, facilitating placement or removal of a spherical ball on the humeral head. FIG. 4 is a side view of the embodiment shown in FIG. 1; FIG. 5 is another side view of the embodiment shown in FIG. 1, also illustrating channel 124 of the reverse adapter aperture 120 extending within the neck 106 and stem 110. FIG. 6 is an inverted perspective view, also illustrating medial groove 126 along the neck 106 and stem 110 of the prosthesis 100. FIG. 7 is another perspective view of the prosthesis shown in FIG. 1.

FIGS. 7A-7G illustrate views of another embodiment of a humeral replacement prostheses somewhat similar to, and which can incorporate any number of features shown, for example in FIGS. 1-7. FIG. 7A illustrates a perspective view of the prosthesis 700 including optional proximal ring element 702, such as a collar, with a peripheral edge 703 that can be annular such as circular as shown, with an inferior-facing surface that can be optionally recessed (including a convex or flat bowl-like shape) and include a cavity, or inline in other embodiments. The humeral prosthesis 700 can include a superior-facing surface 714 of the proximal end 706, and be concave and bowl-shaped as shown with the base and deepest portion of the bowl proximate the center of the superior facing surface 714 of the proximal end 106 such that there is a space 713 between the inferior-facing surface of the humeral head portion (not shown) and the superior-facing surface 714 of the proximal end 706 of the prosthesis 700. The proximal end 706 of the prosthesis 700 can include spaced-apart flanges 707, such as three flanges 707 spaced equally apart, with vertices 711 of the flanges 707 extending radially outwardly while becoming narrower from the center of the proximal end 706 of the prosthesis 700. Extending distally from the proximal end 106 at an angle to the longitudinal axis of the proximal end/neck 706 is the stem 710 and distal end 708 of the prosthesis 700. Also illustrated is transition zone 747, of which proximal to that zone 747 the prosthesis 700 includes a porous coating, and distal to that zone 747 the prosthesis 700 does not include a porous coating. The coating could include, for example, a plasma spray, porous metal, hydroxyapatite, or other component which can facilitate cementless fixation to bone. However, cement fixation can be utilized in some embodiments.

FIG. 7A is a side perspective view of an embodiment of the prosthesis; FIG. 7B is a bottom perspective view of the embodiment shown in FIG. 7A; FIG. 7C is a top view of the embodiment shown in FIG. 7A also showing an aperture 120 in the center of the superior facing surface 114 of the neck 106 of the prosthesis and configured to be utilized as a reverse adapter for, in some cases, facilitating placement or removal of a spherical ball on the humeral head. FIG. 7D is a side view of the embodiment shown in FIG. 7A; FIG. 7E is another side view of the embodiment shown in FIG. 7A, also illustrating channel 124 of the reverse adapter aperture 120 extending within the neck 106 and stem 110. FIG. 7F is an inverted perspective view, also illustrating medial groove 126 along the neck 106 and stem 110 of the prosthesis 100. FIG. 7G is another perspective view of the prosthesis shown in FIG. 7.

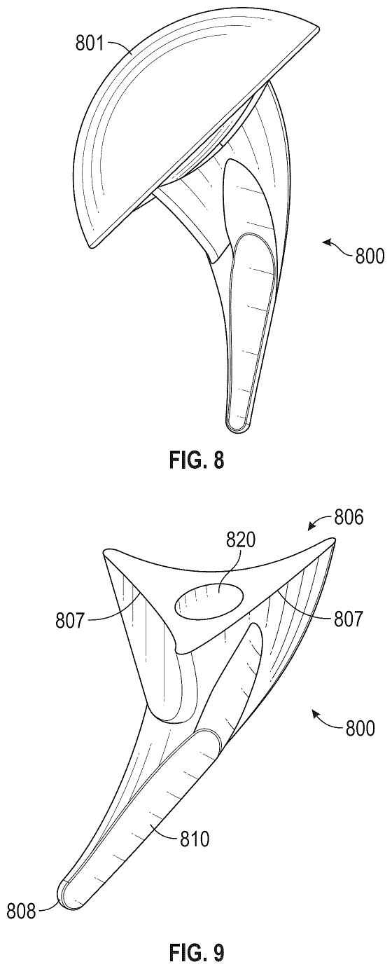

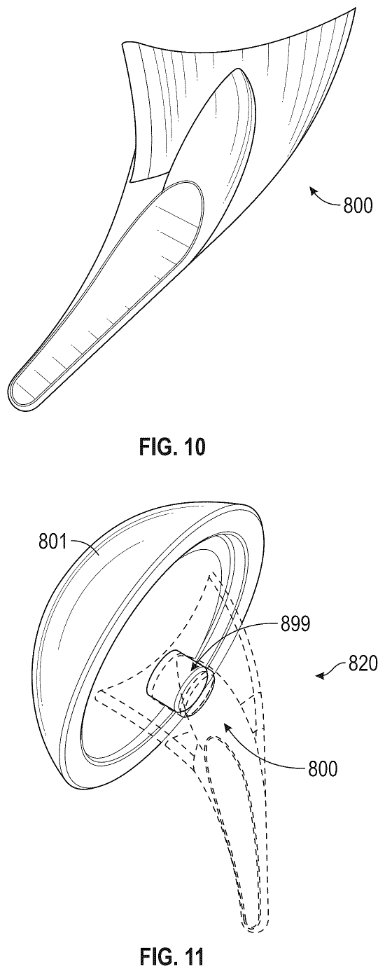

FIGS. 8-11 illustrate various views of another embodiment of a humeral replacement prosthesis. FIG. 8 illustrates a humeral prosthesis that can be as described elsewhere herein, configured to be connected to a humeral head 801. FIG. 9 illustrates a perspective view of the humeral prosthesis 800, including stem 806, flanges 807, and adapter aperture 820 on the proximal end 806, as well as distal end 808. FIG. 10 illustrates a side view of the humeral prosthesis 800. FIG. 11 illustrates humeral head prosthesis 801 configured to be connected via projection 899 to the adapter aperture 820 of the humeral prosthesis 800, such as via press fit, threads, adhesive, or other techniques.

As shown, some potentially advantages of humeral implant configurations as described herein include that fixation is concentrated proximally, to conserve native bone. The prosthesis could be cemented, or cementless in some embodiments. The prosthesis geometry allows for ease of use, minimized surgical steps, and robust as errors can be readily absorbed. The prosthesis can also be convertible to reverse, which increases potentially for success and prevents or minimizing overstuffing. The prosthesis can also be advantageously inexpensive, logistically simple, and have minimal inventory requirements, as a diameter at or near the distal end of the stem can be constant, while the diameter at or near the proximal end of the stem can vary. For example, an operator could select a small number of humeral stem implants, e.g., with 34 mm, 38 mm, and 44 mm proximal diameters (which can be measured as the smallest circle that can encircle the proximal-most radially-outward end elements of the fins), all of which have the same distal-most diameter, such as about 5.5 mm or about 6 mm, or no more than about 7 mm, 6.5 mm, 6 mm, 5.5 mm, 5 mm, or less, or ranges including any two of the foregoing values. In some embodiments, the proximal-most diameter can be between about 25 mm and about 50 mm, such as about 25, 30, 35, 40, 45, 50 mm, or ranges including any two of the foregoing values. In some embodiments, the humeral stem implants can have a ratio of proximal-most to distal-most diameter of the humeral stem of between about 5 and about 9, between about 5.5 and about 8.5, about 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, or ranges including any two of the foregoing values. The ability to provide a set or array of humeral stem implants that a physician can choose from with varying proximal-most diameters, each with the same or substantially the same small distal-most diameter can be in contrast to conventional humeral stem prostheses, in which within a set of different humeral stem sizes, the distal-most diameter generally also increases as the proximal-most diameter increases. In some embodiments, the prosthesis is collarless. In some embodiments, the prosthesis has a total length of about or at least about 50 mm, 51 mm, 52 mm, 53 mm, 54 mm, 55 mm, 56 mm, 57 mm, 58 mm, 59 mm, 60 mm, 65 mm, 70 mm, or more, or ranges incorporating any two of the aforementioned values.

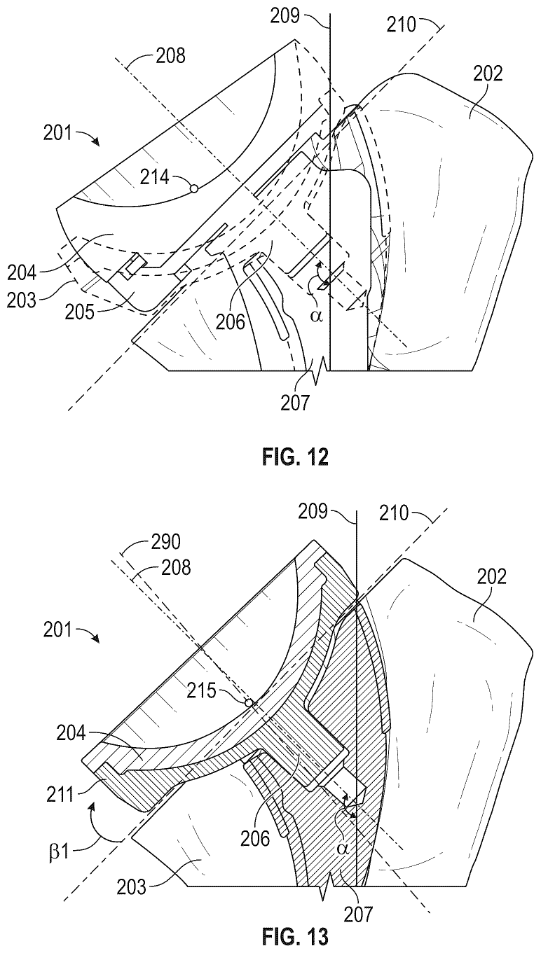

FIGS. 12-15 illustrate various views of several embodiments of components of a glenohumeral replacement prosthesis. FIG. 12 can be used to compare the effects of several different embodiments of the invention as depicted in FIGS. 13-15.

FIG. 12 depicts a conventional shoulder implant component embedded into the fossa of the scapula 201, such as the glenoid, or a humerus for example. FIG. 12, depicts an implant with an inclined, offset bearing component 204 (referring to the central axis of the bearing component being offset from the central axis of the backing component), with a backing component 205 which is aligned with center of rotation 208. The prosthetic is embedded on or within the bony cavity 203 comprises a bearing surface 204 embedded onto a backing component (e.g., a concave tray) 205. The bearing surface 204 lines the backing component 205 and can be made of any appropriate material, such as a non-metallic, biologically compatible material (e.g. polyethylene). The backing component connects to an elongated stem 207 that is embedded into the bone 202. The backing component 205 connects with the elongated stem 207 by a peg or keel (e.g. a morse taper) 206. In FIG. 12, the stem has neck shaft angle of about 132.5 degrees a which is the angle created by intersection of the axis through the elongated stem 209 and the center of rotation 208. The backing component 205 is aligned with the axis of the fossa 210. Since the bearing component 204 is offset, the center of rotation 208 of the glenosphere 214 is medialized.

FIG. 13 illustrates a different embodiment wherein the inclination angle of the humerus angle is controllable exclusively by an offset backing component 211, while the bearing component is not offset. By offset, the peg 206 of the backing component 211 is positioned such that a first distance from the peg 206 to a first end of the offset backing component 211 is not equal to a second distance from the peg 206 to a second send of the offset backing component 211. In some embodiments, the first distance is at least about, about, or no more than about 5%, 10%, 15%, 20%, 25%, or more or less than the second distance, or ranges including any two of the foregoing values. This is in distinct contrast to FIG. 12 wherein the bearing component (e.g., polyethylene) is offset with respect to a central axis of the backing component. FIG. 13 shows the effect of a reverse articulation at 135 degrees using a backing component that provides a 2.5 degree incline 211 when attached to the elongated stem 207. The rotation β1 represents a rotation of 2.5 degrees about the axis of the glenoid fossa 210. When added to the angle α, a reverse articulation of 135 degrees is achieved. Since the bearing component is not offset as in FIG. 12, the center of rotation of the glenosphere 215 remains constant, and is not medialized (or lateralized) away from the center of rotation 208. Also, the central axis 290 of the backing component 211 is offset from the central axis 208 of the peg.

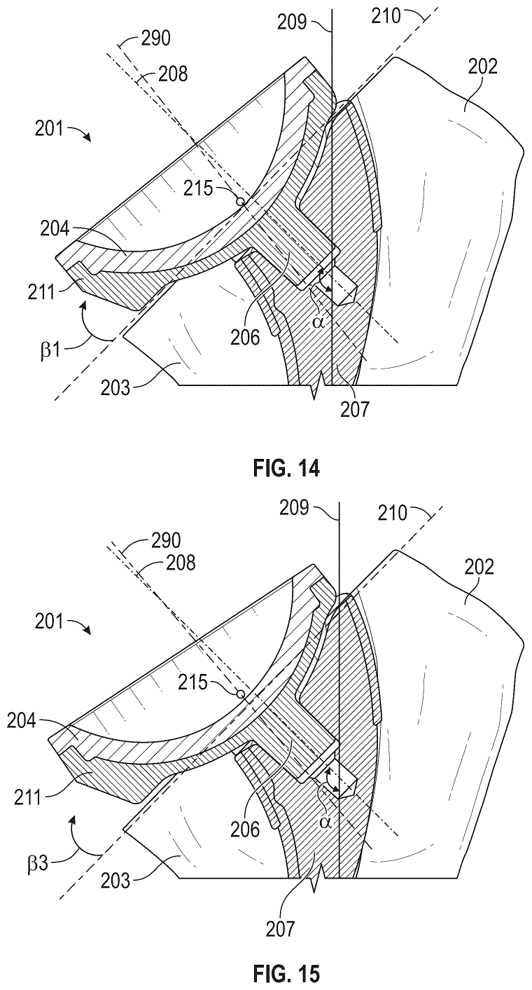

FIG. 14 is similar to FIG. 13 with the exception that the angle of inclination of the backing component is different. FIG. 14 shows a desired 140 degree angle with a backing component providing a 7.5 degree incline 212 when attached to the elongated stem 207. The rotation β2 represents a rotation of 7.5 degrees about the axis of the glenoid fossa 210. When added to the angle α, a reverse articulation of 140 degrees is achieved. Since the bearing component is not offset as in FIG. 12, the center of rotation of the glenosphere 215 is not medialized away from the center of rotation 208 and remains in the same position as it was in FIG. 13.

FIG. 15 illustrates a 145 degree angle with a backing component providing a 12.5 degree incline 213 when attached to the elongated stem 207. The rotation β3 represents a rotation of 12.5 degrees about the axis of the glenoid fossa 210. When added to the angle α, a reverse articulation of 145 degrees is achieved. Since the bearing component is not offset as in FIG. 12, the center of rotation of the glenosphere 215 is not medialized away from the center of rotation 208 and remains in the same position as it was in FIG. 13 and FIG. 14.

In some embodiments of the invention, the inclination angles produced by the backing component above and beyond the neck shaft angle of the elongated stem may range from about 1.25, 2.5, 3.75, 5, 7.5, 10, 12.5, 15, 17.5, 20, 22.5, 25, 27.5, 30, 32.5, 35 degrees, or ranges including any two of the aforementioned values.

The native prepared surface of the humerus of which an inferior-facing surface of the backing component rests against after implementation generally has an oval or elliptical cross-section. In some embodiments, the inferior-facing surface of the backing component also has an oval or elliptical cross-section substantially matching that of the surface of the humeral bone. In some embodiments, at least a portion of the inferior facing surface of the bearing component (e.g., polyethylene component in some cases) has a circular cross-section, and is configured to snap into, or otherwise mate with a complementary portion of the superior-facing surface of the backing component, which can also have a circular cross-section.

Non-limiting potential advantages of the embodiments in FIG. 13-15 is observed by example when comparing FIG. 12 to FIG. 14. The depth of the backing component in FIGS. 13-15 can be about 5 mm, compared with about 7.5 mm with respect to the FIG. 12 embodiment where the bearing component is offset with respect to the center of rotation of the glenosphere, as well as the central axis of the backing component. In some embodiments, the depth of the backing component is less than about 6, 5.5, 5, 4.5, 4, 3.5, 3 mm or less, or ranges including any two of the aforementioned values. Changing the backing component angle in FIGS. 13-15, as opposed to changing the amount of polyethylene plastic or other material in the bearing component, results in an additional amount of space, such as about 2.5 mm or more or less of space formed in the bowl for rotation of the glenosphere. Therefore, in several of the embodiments presented, the joint is less “stuffed” and not overstuffed and can potentially have greater range of motion. Furthermore, a physician can select between a range of different backing components with varying inclination angles, while the bearing components are neutral (e.g., not inclined). As such, the inclination angle of the humerus can be controlled solely by selecting an appropriate backing component, and the center of rotation of the glenosphere remains constant irrespective of the potentially varying inclination angle of the backing component.

In some embodiments, a glenohumeral implant allowing for adjustable angles for anatomic or reverse arthroplasty is presented and comprises: a backing component; a bearing component on the concave side of the backing component, the bearing component made from a different material than the backing component; and a peg extending from the backing component. Further, in some embodiments the longitudinal axis of the backing component is offset from the center of rotation, but the center of rotation is not changed.

In some situations, it is desirable to avoid using a stemmed humeral implant. Unlike a stemmed humeral implant, a stemless humeral implant has a shorter overall length such that no part of the implant enters the canal of the humerus when implanted. Instead, fixation occurs totally within the cancellous portion of the bone. The stemless implants fins do not enter the canal of the bone, and direct marrow displacement does not occur during implantation. Instead, fixation is limited to the metaphyseal portion of the bone, sometimes referred to as the proximal region of the humerus. A small portion of the distal end of the stemless humeral implant may enter the diaphysis portion of the bone, but in no case will the implant extend to the intramedullary canal (where the marrow is located).

The stemless implant can advantageously provide at least equal fixation strength as a stemmed implant but with a less invasive procedure and with less removal of native bone structure, as discussed below. The stemless implant can simplify implantation because there is no need to determine the diameter of the patient's humerus in order to properly size an implant stem (in length or diameter). Instead, the stemless implant is press fit into the humerus proximal region without disrupting the rest of the humerus.

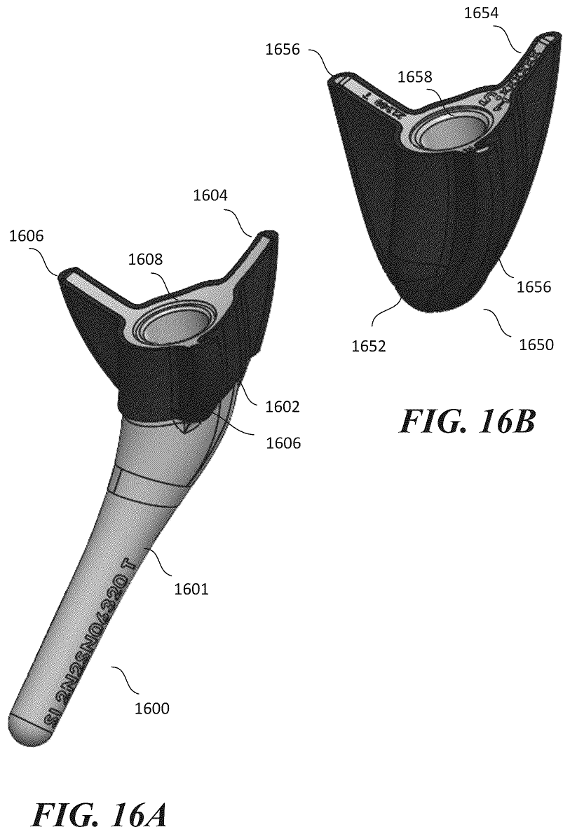

FIG. 16A and FIG. 16B show a perspective view of a stemmed humeral implant 1600 and a stemless humeral implant 1650, respectively. Each implant 1600, 1650 has a base portion (sometimes referred to as a backing component) 1602, 1652 that includes a plurality of fins (or flanges) 1604, 1606, 1654, 1656. The stemmed humeral implant 1600 includes a stem 1601. The illustrated embodiments each include one lateral fin 1604, 1654 and two medial fins 1606, 1656. The implants 1600, 1650 are symmetric about a plane that bisects the lateral fin 1604, 1654 along its length (from the implant top or proximal end to the implant bottom or distal end). However, the fins 1604, 1606, 1654, 1656 need not be symmetric about such plane, and indeed, may each have different dimensions and orientations.

Both implants 1600, 1650 include an opening 1608, 1658 that is sized to receive and secure a base (not shown) (sometimes referred to as a non-inclined bearing component or a neutral non-inclined bearing component). The opening may include a slight taper, e.g., a Morse taper, to enable a taper fit, or press fit, between the implant 1600, 1650 and base. The base may include an articulation surface, such as a glenosphere (for anatomic implantation) or a concave base (for reverse implantation). Any of the techniques described above for affixing a base (sometimes referred to as a base portion) to an implant 1600, 1650 may be utilized.

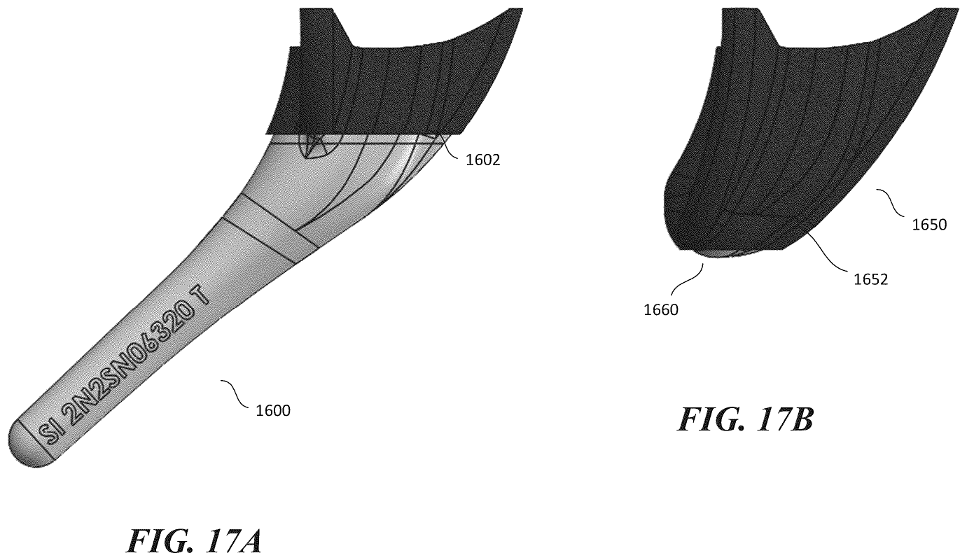

FIG. 17A is a side view of the stemmed humeral implant 1600 of FIG. 16A and FIG. 17B is a side view of the stemless humeral implant 1650 of FIG. 16B. The base portions 1602, 1652 may be covered with a bone-growth-promoting material, such as a porous coating, or any of the materials described herein. In one embodiment, the stemless implant 1650 does not include any bone growth material at its distal tip 1660. Providing an uncoated tip 1660 can facilitate implant 1650 removal if needed. The base portions 1600, 1650 may include any of the coatings described herein. For example, the coating could include, for example, a plasma spray, porous metal, hydroxyapatite, or other component which can facilitate cementless fixation to bone. In other embodiments, the coating may include an osteoinductive or osteoconductive surface to facilitate bone ingrowth and fixation into the cavity. The surface could include, for example, cortical bone, cancellous bone, particulate matter, a powder form, granules, chips, a synthetic bone substitute, growth factors and/or bone growth promoting proteins, or combinations thereof.

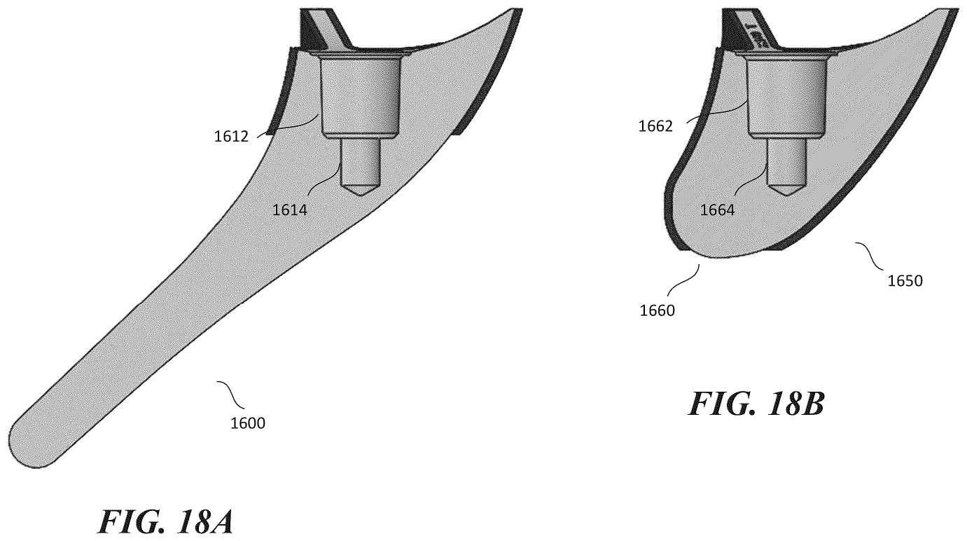

FIG. 18A is a cross-section view of the stemmed humeral implant 1600 of FIG. 16A and FIG. 18B is a cross-sectional view of the stemless humeral implant 1650 of FIG. 16B. Each implant 1600, 1650 includes a tapered cavity 1612, 1662 and a threaded cavity 1614, 1664. The tapered cavity 1612, 1662 is sized and tapered to receive a base having either an anatomic or reverse articular surface attached thereto. The base (not shown) is tapered to provide a taper fit, Morse taper, and/or press fit securement to the tapered cavity 1612, 1662 of the implant 1600, 1650. The threaded cavity 1614, 1664 may be used to secure an instrument to remove the base (not shown), if desired. For example, by using the threaded cavity 1614, 1664, the base may be removed to replace an anatomic base with a reverse base, as described in greater detail above.

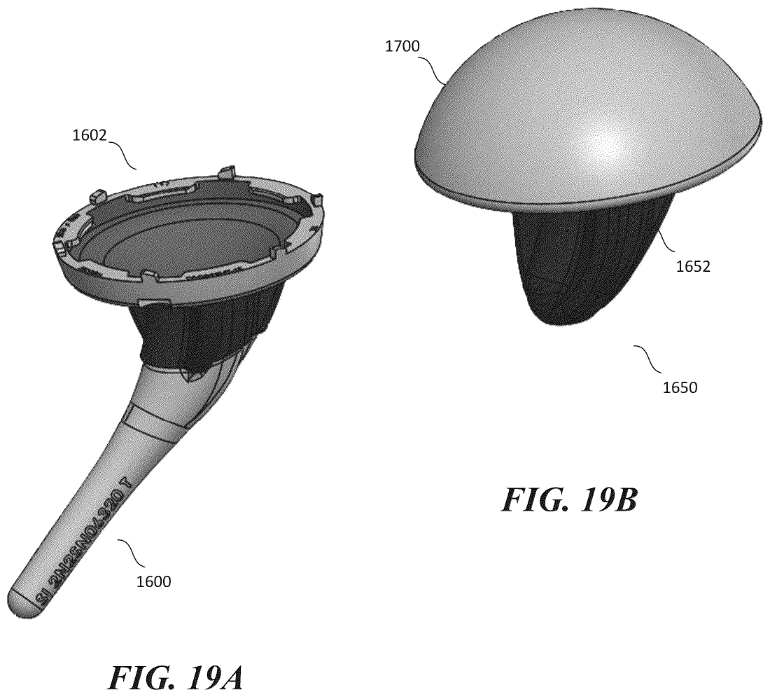

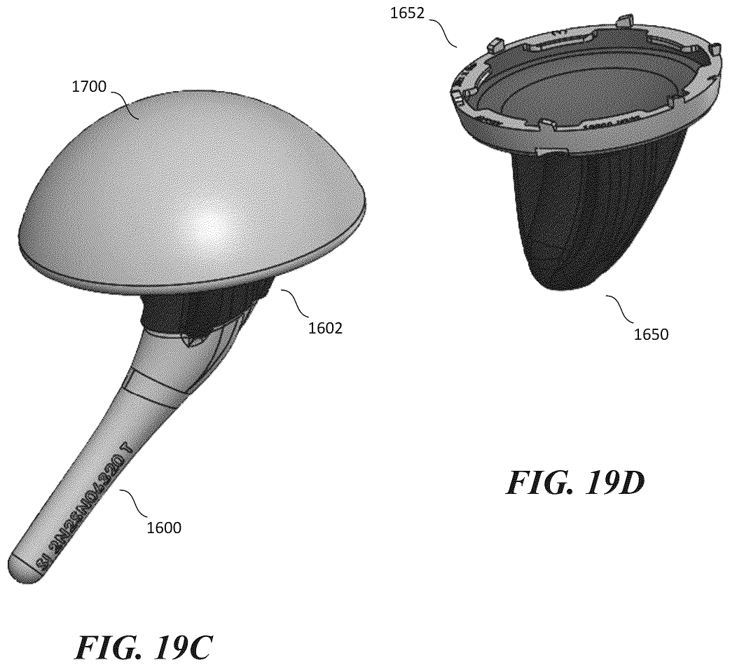

FIGS. 19A through 19D illustrate the compatibility between the stemmed and stemless humeral implants 1600, 1650 by showing that a glenosphere 1700 attached to a base portion 1652 of a stemless humeral implant 1650 (as shown in FIG. 19B) is sized to be attached to a base portion 1602 of the stemmed humeral implant 1600, as well (as shown in FIG. 19C). FIG. 19A shows a stemmed humeral implant 1600 without a glenosphere 1700 and FIG. 19C shows the stemmed humeral implant 1600 with the glenosphere 1700. FIG. 19B shows the stemless humeral implant 1650 with the glenosphere 1700, and FIG. 19D shows the stemless humeral implant 1650 without the glenosphere 1700.

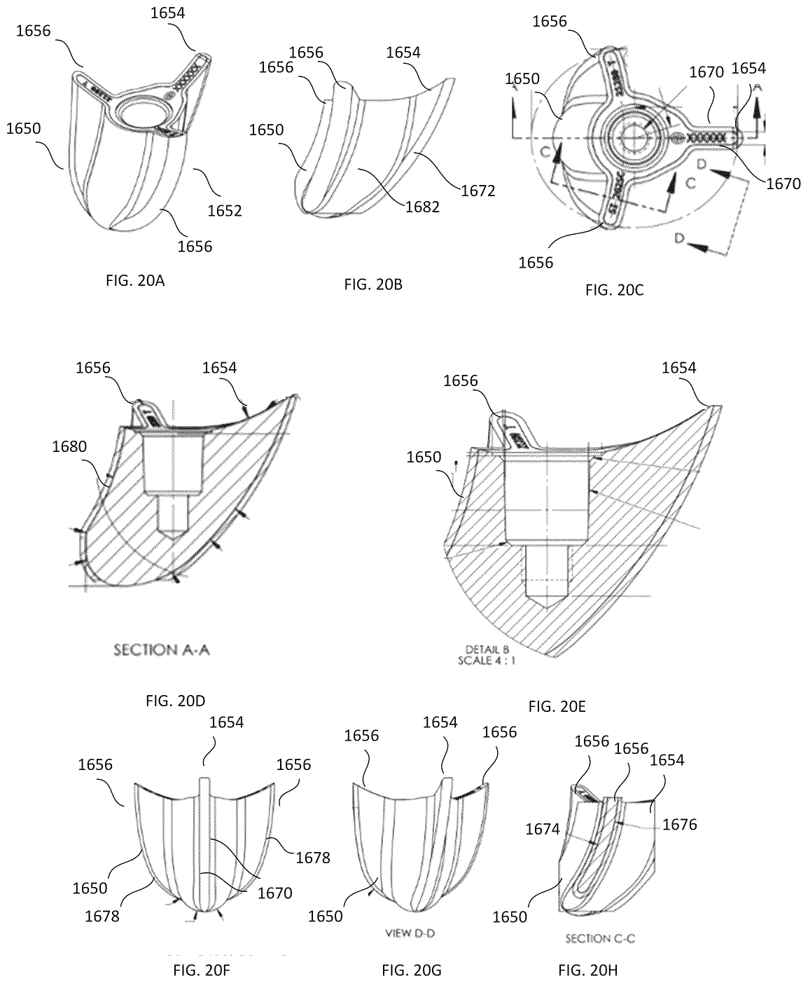

FIGS. 20A-20H show various views of one embodiment of a stemless implant 1650, as shown in FIG. 16B. The stemless implant 1650 includes a plurality of fins, such a three or more fins. The illustrated embodiment includes a lateral fin 1654 and two medial fins 1656. Each fin 1654, 1656 has two sides and an outer edge. The lateral fin's 1654 sides 1670 are flat and may be parallel to each other (see FIGS. 20C and 20F) and its outer edge 1672 has a convex curvature (see FIGS. 20A, 20B, 20D, 20E, and 20G). The medial fins 1656 (sometimes referred to as anterior and posterior fins) each have a concave surface 1674 and an opposing convex surface 1676 (see FIGS. 20A and 20H). Each medial fin's 1656 convex surface 1676 is closest to and faces towards the direction of the lateral fin 1654 (see FIGS. 20A, 20H). Each medial fin's 1656 outer edge 1678 has a convex curvature. In one embodiment, the curvature of the medial fins 1656 and the stemless implant body 1652 may be defined in reference to a pivot or radius spaced equally from the medial fins 1656 that defines an overall radius of curvature of the stemless implant 1650. The portion 1680 of the implant body 1652 between the medial fins 1656 has a concave curvature (see FIG. 20D) and the portion 1682 of the implant body 1652 between the medial fins 1656 and the lateral fin 1654 has a convex curvature (see FIGS. 20A, 20B, 20F, 20G, and 20H).

The curvature of the fins 1654, 1656 provides for greater lever out loads than implants having only straight fins, or fins with flat sides. The curvature of the fins 1654, 1656 and body 1652 makes it much stronger, and more difficult to pry out of bone, thereby providing much improved initial and long-term fixation. The overall length of the implant 1650, as defined from the most lateral proximal point of the stemless implant 1650 to the most distal point of the implant 1650 is no longer than 40 mm. In addition, when viewed from above, the outermost edges of the fins 1654, 1656 may fall within or are enclosed by an oval or ellipse drawn to surround the implant 1650. In one embodiment, the outermost edges of the fins 1654, 1656 do not fall within and are not enclosed by a circle drawn to surround the implant 1650. The radial dimension of each fin 1654, 1656 tapers and decreases from its proximal end to its distal end, as well. The taper allows the implant 1650 to be inserted into the bone without perforating the bone's side.

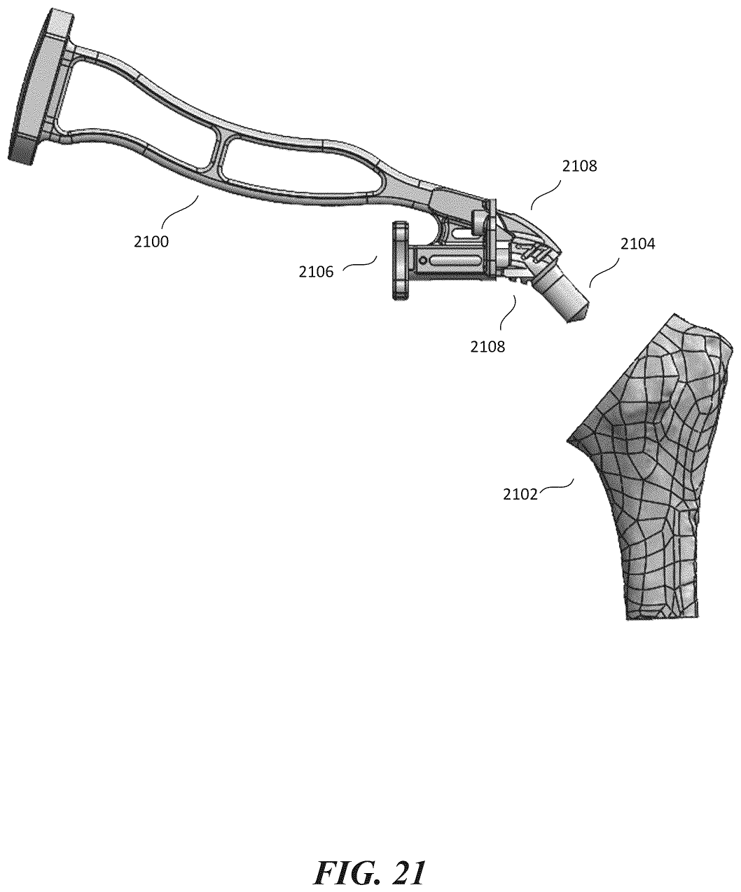

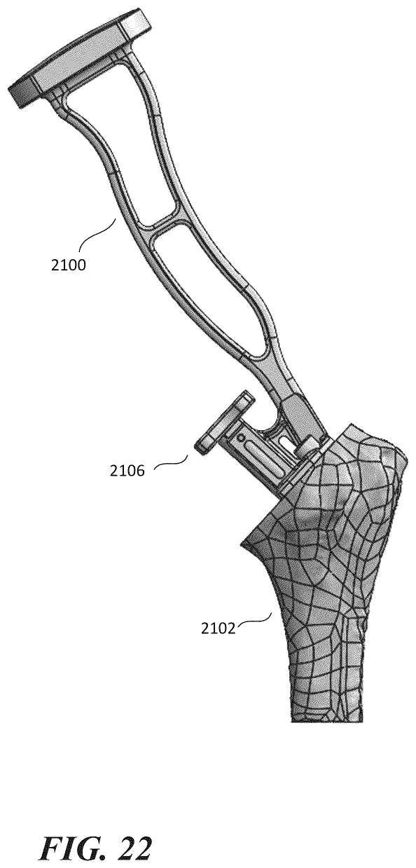

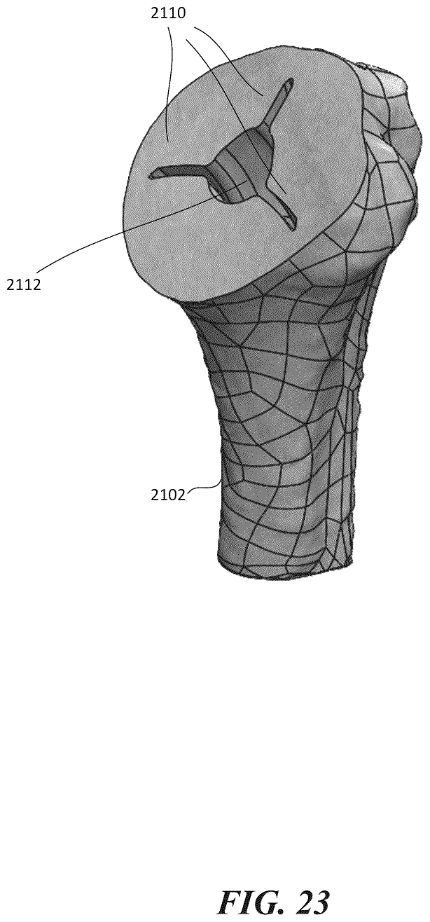

FIG. 21 illustrates one embodiment of a broach 2100 that may be used to prepare a humerus 2102 to receive a stemmed or stemless humeral implant. An alignment post 2104 is positioned at a desired, central location on the proximal face of the humerus 2102. A hammering force is applied to an impact plate 2106 to drive the broach's cutting fins 2108 into the cancellous bone at the metaphysis of the humerus 2102, as shown in FIG. 22. When the broach 2100 is removed, the cancellous bone has narrow slots 2110 configured to receive the fins of the stemmed or stemless implant, and a circular and/or cylindrical opening 2112 configured to receive the implant body.

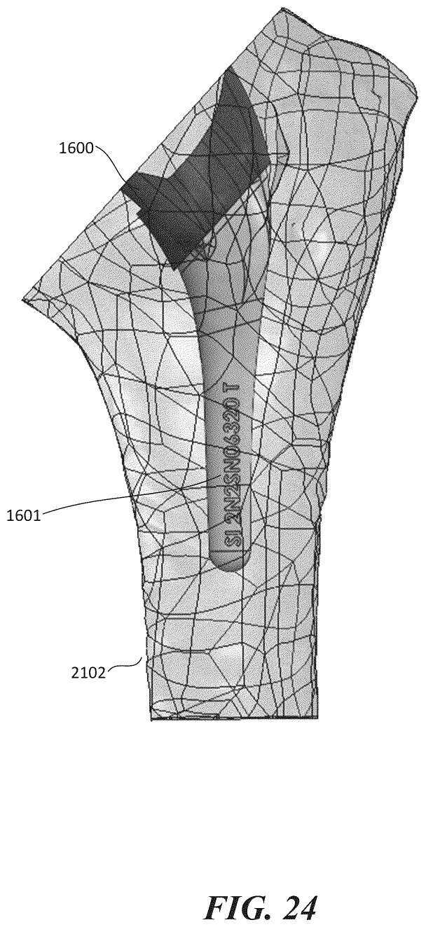

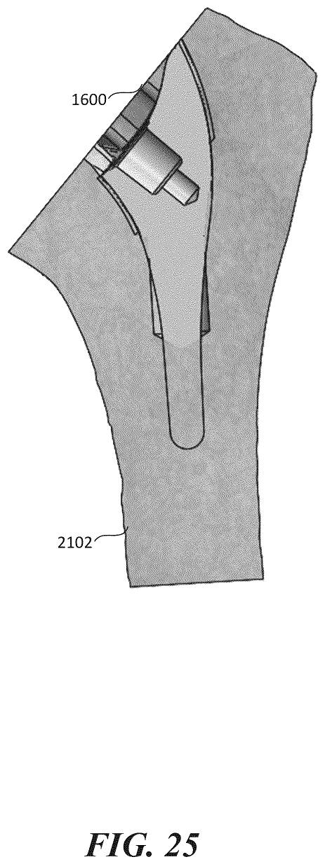

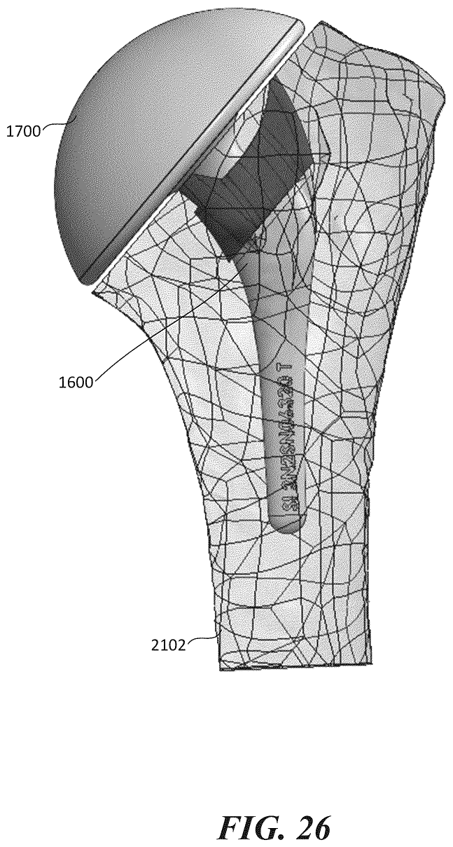

FIG. 24 shows a stemmed implant 1600 implanted into a humerus 2102. The stem 1601 of the stemmed implant 1600 extends into the bone's 2100 canal. FIG. 25 shows a cross sectional view of the implant 1600 of FIG. 24, implanted into a humerus 2102. FIG. 26 shows the implant 1600 of FIG. 24 having an articular surface, in this case a glenosphere cap 1700, attached to the stemmed implant 1600.

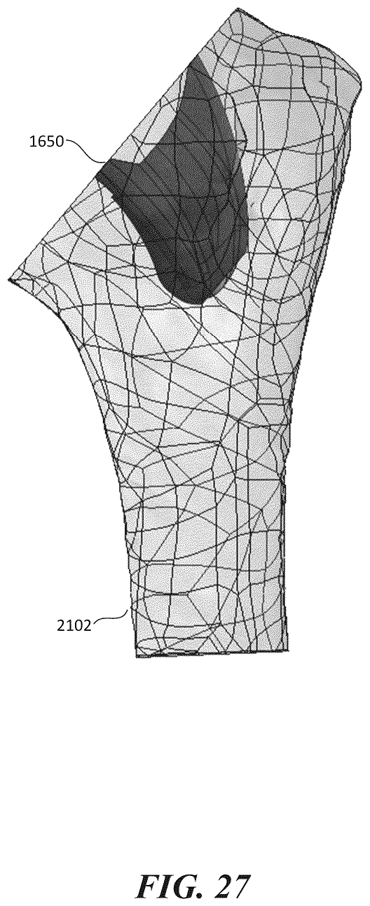

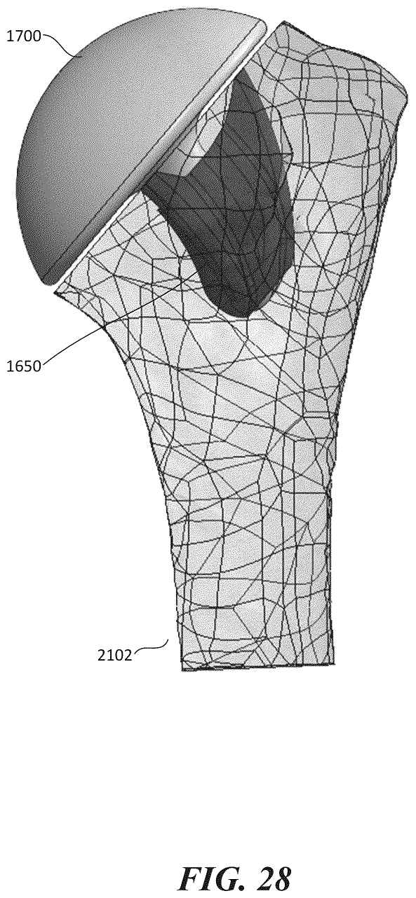

FIG. 27 shows a stemless implant 1650 implanted into a humerus 2102. The stemless implant 1650 does not extend into the bone's 2102 canal. Instead, it is fixed solely to the metaphysis portion of the humerus 2102. FIG. 28 shown the stemless implant 1650 of FIG. 27 having an articular surface, in this case a glenosphere cap 1700, attached to the stemless implant 1650.

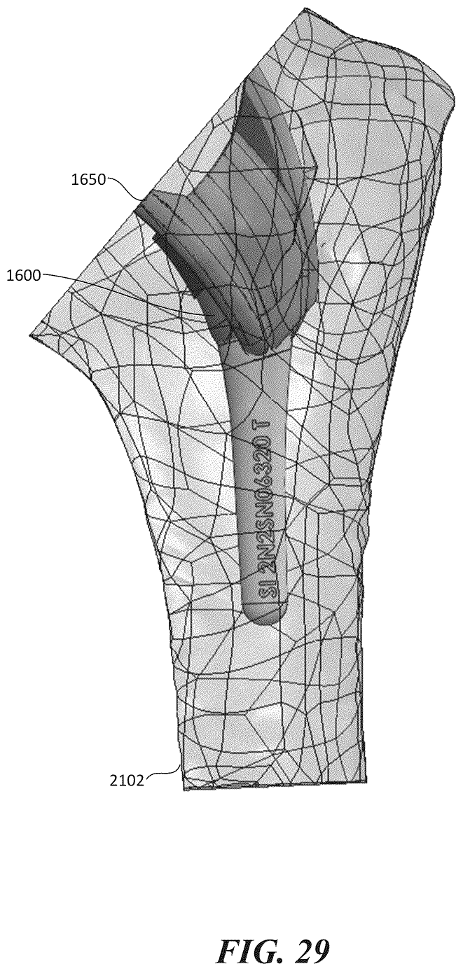

FIG. 29 shows what it would look like if both a stemmed and a stemless implant 1600, 1650 were inserted into the same humerus 2102. The illustration is provided for reference and comparative purposes only.

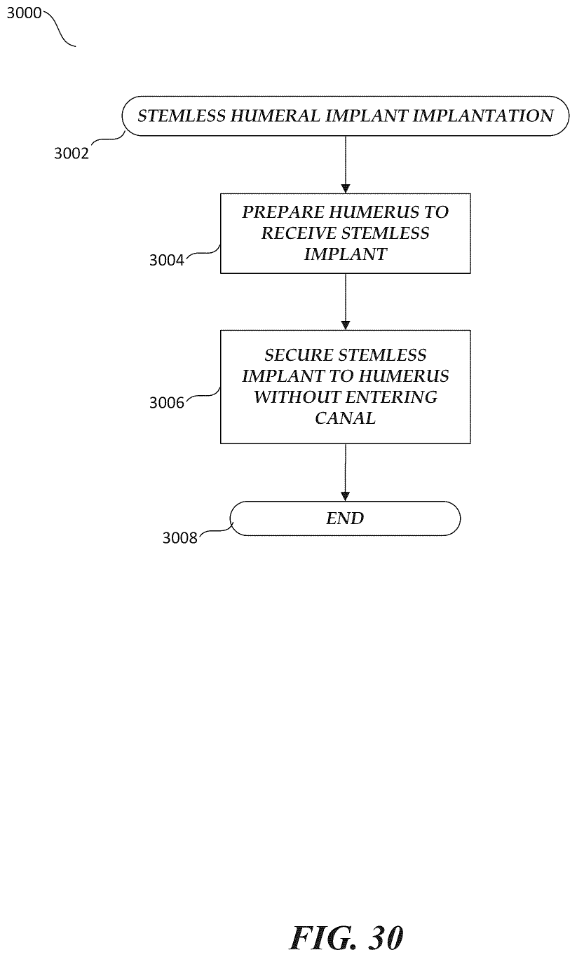

FIG. 30 is one embodiment of a method 3000 of implanting a stemless implant. The method 3000 begins at block 3002. A humerus is prepared to receive a stemless implant at block 3004. For example, a broach may be used to cut slots and a cylindrical opening within the metaphysis of the humerus. At block 3006, a stemless implant is then secured to the humerus. For example, fins of the stemless implant may be inserted into the slots cut by the broach, and the body of the stemless implant may be inserted into the cylindrical opening formed by the broach. Fixation occurs solely at the metaphysis, as no portion of the implant is inserted into the humerus inner canal. The method 3000 ends at block 3008.

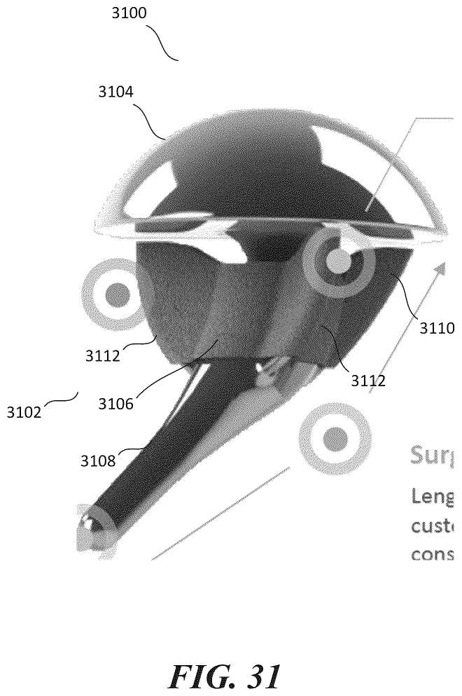

FIG. 31 shows one embodiment of a stemmed humeral implant system 3100. The stemmed humeral implant system 3100 includes a stemmed humeral implant 3102 and an articular surface, in this case, a glenosphere 3104. The stemmed humeral implant 3102 includes a base portion 3106 and a stem 3108. The base portion 3106 includes one lateral fin 3110 and two medial fins 3112.

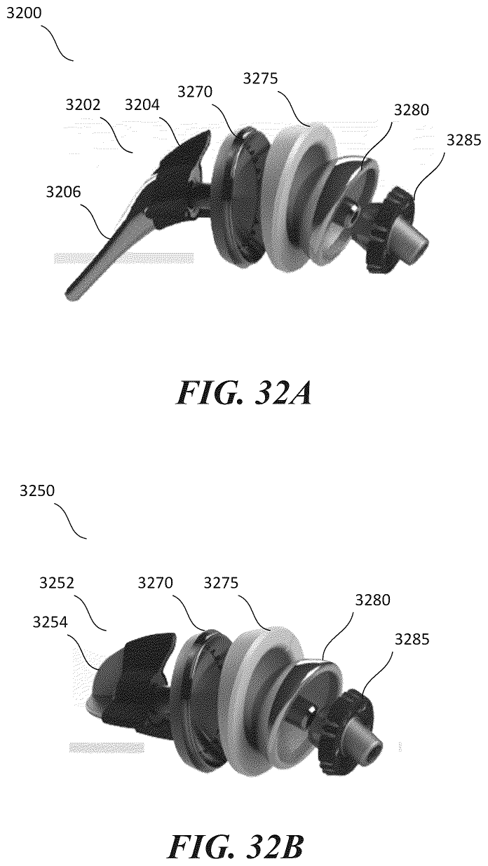

FIG. 32A and FIG. 32B show exploded views of a stemmed humeral implant system 3200 and a stemless humeral implant system 3250, respectively. The stemmed humeral implant system 3200 includes a stemmed humeral implant 3202. The stemmed humeral implant 3202 includes a base portion 3204 (sometimes referred to as a backing component) and a stem 3206. The stemless humeral implant system 3250 includes a stemless humeral implant 3252. The stemless humeral implant 3252 includes a base portion 3255 (sometimes referred to as a backing component).

Both the stemmed humeral implant system 3200 and the stemless humeral implant system 3250 include bases 3270 (sometimes referred to as a non-inclined bearing component or a neutral non-inclined bearing component) that are removably attachable to their respective base portions 3204, 3254. The non-inclined bearing component may be located on a concave side of the backing component and made from a different material than the backing component. The base 3270 is configured to receive an articulating surface 3270, such as a concave articulating surface (as shown) or a glenosphere. The articulating surface 3270 is configured to interface with a mating articulating surface, such as a glenosphere 3280. A fixing member 3285 is provided to attach the mating articulating surface (e.g., the glenosphere 3280, in the illustrated example) to scapular structure.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “insetting an implant into a glenoid cavity” includes “instructing the insetting of an implant into the glenoid cavity.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A shoulder implant with improved joint mobility, comprising:
a backing component;
a neutral non-inclined bearing component on a concave side of the backing component, the bearing component made from a different material than the backing component and configured to touch a glenosphere having a center of rotation, wherein the bearing component is substantially radially symmetrical; and
a peg extending from the backing component and configured to be connected to a humeral stem;
wherein the longitudinal axis of the peg of the backing component is offset from the center of rotation of the glenosphere,
wherein the longitudinal axis of the backing component is aligned with the center of rotation of the glenosphere.

2. The shoulder implant of claim 1, wherein the backing component is inclined by an angle of between about 1.25 degrees and about 30 degrees with respect to a corresponding attachment surface of a humeral head.

3. The shoulder implant of claim 1, wherein the backing component is inclined by an angle of between about 2.5 degrees and about 12.5 degrees with respect to a corresponding attachment surface of a humeral head.

4. The shoulder implant of claim 1, wherein the glenoid implant when inserted into a patient does not change the center of rotation of the glenosphere.

5. The shoulder implant of claim 1, configured for anatomic positioning.

6. The shoulder implant of claim 1, configured for reverse positioning.

7. The shoulder implant of claim 1, wherein the bearing component has a depth of less than about 5.5 mm.

8. The shoulder implant of claim 1, wherein the bearing component has a depth of between about 3 mm and about 5 mm.

9. The shoulder implant of claim 1, wherein the bearing component comprises a plastic material.

10. The shoulder implant of claim 9, wherein the plastic material comprises polyethylene.

11. The shoulder implant of claim 1, wherein the backing component comprises a metal material.

12. The shoulder implant of claim 1, wherein the peg comprises a Morse taper.

13. The shoulder implant of claim 1, wherein at least a portion of an inferior-facing surface of the backing component configured to come in contact with a prepared humeral surface comprises an oval or elliptical cross-section, at least a portion of the inferior-facing surface of the bearing component comprises a circular cross-section, and at least a portion of the superior-facing surface of the backing component comprises a circular cross-section, the portion of the inferior-facing surface of the bearing component and the superior-facing surface of the backing component configured to snap or otherwise mate with each other.

14. A kit of shoulder implants, comprising a plurality of implants as in claim 1, wherein the plurality of implants comprises a first implant and a second implant, wherein the backing component of the first implant is inclined by a first angle, wherein the backing component of the second implant is inclined by a second angle different from the first angle, wherein the center of rotation of the glenosphere of the first implant and the second implant are in the same location.

15. A method of implanting a shoulder implant, comprising:
reaming a cavity in the glenoid surface;
sizing a first implant comprising a first angulation with respect to a stem, wherein the implant comprises a neutral bearing component and an inclined backing component comprising a first incline angle with respect to a corresponding attachment surface of a humeral head;
sizing a second glenoid implant comprising a second angulation with respect to the stem, wherein the glenoid implant comprises a neutral bearing component and an inclined backing component comprising a second incline angle with respect to a corresponding attachment surface of a humeral head;
implanting the second glenoid implant in the glenoid cavity;
wherein the center of rotation of the glenosphere does not change between the first glenoid implant and the second glenoid implant, allowing for adjustment of version and inclination without changing the center of rotation.

16. A humeral prosthesis, comprising:
a proximal end comprising at least three radially outwardly extending fins;
a stem extending distally from the proximal end to a distal end,
wherein the proximal end comprises a proximal end diameter defined by a circle contacting radial outward-most proximal tips of the at least three radially outwardly extending fins,
wherein the distal end comprises a distal end diameter,
wherein the proximal end diameter is between about 30 mm and about 50 mm,
wherein the distal end diameter is between about 5 mm and about 6 mm, and
wherein a ratio of the proximal end diameter and the distal end diameter is between about 5 and about 9.

17. The humeral prosthesis of claim 15, wherein the ratio is between about 5.5 and about 8.5.

18. The humeral prosthesis of claim 15, wherein the proximal end comprises a bowl-shaped concavity.

19. The humeral prosthesis of claim 15, comprising a central aperture in the proximal end configured to mate with a reverse component.
